(19) United States
(12) Patent Application Publication
(10) Pub. No.: US 2002/0142338 A1
(43) Pub. Date: Oct. 3, 2002

(54) Compositions and methods for inhibiting motor neuron degeneration

(76) Inventors: William W. Schlaepfer, Philadelphia, PA (US); Rafaela Canete-Soler, Philadelphia, PA (US)

Correspondence Address: LICATLA & TYRRELL P.C., 66 E. MAIN STREET, MARLTON, NJ, 08053, US

(21) Appl. No.: 10/082,032

(22) Filed: Feb. 21, 2002

Related U.S. Application Data
(63) Continuation-in-part of application No. 09/994,420, filed on Nov. 27, 2001.
Application No. 09/994,420 is a continuation-in-part of application No. 09/489,979, filed on Jan. 21, 2000, now Pat. No. 6,323,391.
(60) Provisional application No. 60/117,007, filed on Jan. 25, 1999.

(51) Int. Cl. C12Q 1/68; C12N 9/64

(52) U.S. Cl. 435/6; 435/226

(57) ABSTRACT

An RNA neuropathic element is provided that is produces motor neuron degeneration in animals. Methods are provided for identifying components of RNA-protein complexes that can prevent motor neuron degeneration.

### INTRODUCTION

This application is a continuation-in-part of U.S. application Ser. No. 09/994,420, filed Nov. 27, 2001, which was a continuation-in-part of U.S. application Ser. No. 09/489,979, filed Jan. 21, 2000, now issued as U.S. Pat. No. 6,323,391, which claims the benefit of priority of U.S. Provisional Application Serial No. 60/117,007, filed Jan. 25, 1999.

This invention was supported in part by funds from the U.S. Government (National Institutes of Health Grant Nos. NS15722 and NS37552) and the U.S. Government may therefore have certain rights in the invention.

### BACKGROUND OF THE INVENTION

Stabilization of neurofilament (NF) mRNAs is a critical phenomena in determining levels of NF expression, axonal size and rate of axonal conduction. By regulating NF mRNA stability, the neuron is able to establish fundamental functional parameters of its own phenotype. The stabilization of NF mRNA and increased levels of NF expression are strongly influenced by the nature of target cell innervation and, presumably, by feedback signals from the target cells to the parent neuron. The feedback signals regulating NF expression arise during a developmental timeframe in which feedback signals from the target cells are also promoting the survival and further development of the parent neuron. The latter phenomena are believed to involve growth factors produced by the target cell which interact with receptors on the parent neuron and prevent the parent neuron from undergoing apoptosis. Neurons that reach and innervate target cells acquire growth factors and survive, while neurons that fail to innervate target cells do not acquire growth factors and undergo apoptosis. Growth factors thereby enable the developing neurons to override an intrinsic program of apoptosis, as exemplified by the inability of the developing neuron to survive when separated from the target cell by nerve transection or when grown in vitro in the absence of growth factors. During the next phase of development (between 0 and 4 weeks of postnatal development), neurons lose their dependence on growth factors for survival, as exemplified by their ability to survive in vitro in the absence of growth factors as well as by their ability to survive a nerve transection (Schwartz et al., J. Neurosci. Res., 1990, 27:193-201). The weaning of neurons of their dependence on growth factors for survival reflects a change in the expression of genes regulating apoptosis, possibly due to the recruitment of a new set of genes which serve to override apoptosis in the absence of growth factors. While the identity of these anti-apoptosis genes are unknown, it is significant that they impart a vital and unique property to the neuron during the same developmental timeframe in which there is dramatic upregulation in the expression of the three NF genes (Schlaepfer, W. W. and Bruce, J., J. Neurosci. Res., 1990, 25:39-49). The dramatic increase of NF expression is due to the stabilization of NF mRNAs (Schwartz et al., J. Biol. Chem. 1992, 267:24596-24600 and Schwartz et al., Mol. Brain Res., 1994, 27:215-220) and is mediated by factors that bind to the NF mRNAs (Ca\check{n}ete-Soler et al., J. Biol. Chem., 1998, 273:12650-12654; and Ca\check{n}ete-Soler et al., J. Biol. Chem., 1998, 273:12655-12661). If these same factors regulate the expression of anti-apoptotic genes that maintain neuronal homeostasis in the absence of growth factors, the factors themselves would be an important vehicle by which to identify the anti-apoptotic gene products that maintain neuronal homeostasis.

The possibility that the same regulatory factors alter post-transcriptional expression of NF genes and gene products maintaining neuronal homeostasis derives from studies of motor neuron degeneration in transgenic mice bearing neurofilament transgenes. Although the pathogenesis of motor neuron degeneration due to expression of a mutant NF-L transgene (Lee et al., Neuron, 1994, 13:975-988) or overexpression of a wild-type NF-L (Xu et al., Cell, 1993, 73:23-33) or NF-H (Cote et al., Cell, 1993, 35-45) transgene in transgenic mice is presently unknown, it has been generally assumed that the neuropathic effects result from expression of NF protein by the transgene. It has been further assumed that the additional expression of NF protein by the transgene causes motor neuron degeneration by disrupting NF assembly or transport in NF-rich motor neurons (Collard et al., Nature, 1995,375:61-64; Bruijn, L. I. and Cleveland, D. W., Neuropathol. Appl. Neurobiol., 1996,22:373-387).

The interpretation that the pathogenesis of experimental motor neuron degeneration is due to alterations in protein function, however, is problematic on several grounds. While NF accumulations are prima facie evidence of disrupted NF transport, they do not indicate whether disrupted transport is a cause, rather than a result, of neuronal degeneration. Accumulation of NFs is a frequent and readily detectable pathological change that does not necessarily lead to a progressive loss of neuronal viability, even with massive accumulations of NFs in motor neurons (Eyer, J. and Peterson, A. C., Neuron, 1994, 12:389-405). Nor is there appreciable loss of neuronal viability from a marked depletion of NFs due to ablation of NF-L (Zhu et al., Exp. Neurol., 1997, 148:299-316) or medium neurofilament subunit (NF-M) (Elder et al., J. Cell Biol., 1998, 141:727-739) or a spontaneous nonsense mutation of NF-L (Yamashaki et al., Lab. Invest., 1992, 66:734-743). Finally, there is the issue of specificity, as to why a widely and abundantly expressed neuronal protein should lead to the selective degeneration of a very small subset of neurons. It is also unclear why experimental motor neuron degeneration occurs from overexpression of a mouse NF-L (Xu et al., Cell, 1993, 73:23-33) or a human heavy neurofilament subunit (NF-H; Cote et al., Cell, 1993, 73:35-46) transgene, but not from overexpression of an NF-M (Wong et al., J. Cell Biol., 1995, 130:1413-1422) or a chimeric NF-H/lacZ (Eyer, J. and Peterson, A. C., Neuron, 1994, 12:389-405) transgene.

More recently, the effects of NF expression on other models of motor neuron degeneration have been examined by cross breeding transgenic lines of mice. These studies have shown that neither the time-course nor neuropathological effects of primary sensory neuronal degeneration in (wst/wst) wasted mice or primary motor neuron degeneration in SOD-1G37R mutant mice are altered by the presence of a mutant NF-H transgene (NFH/lacZ) causing massive maldistribution of NFs within the afflicted neurons (Eyer et al., Nature, 1998, 391:584-587). On the other hand, the additional expression from a wild-type, full-length human NF-H transgene was found to prolong the lifespan and reduce the neuropathologic effects on motor neurons of the same SOD-1G37R transgenic mice (Couillard-Despres et al., Proc. Nat'l Acad. Sci. USA, 1998, 95:9629-9630). Motor neuron degeneration by an SOD-1 transgene was also slowed by ablation of the endogenous NF-L gene, thereby markedly reducing NF expression (Williamson et al., Proc. Nat'l Acad. Sci USA, 1998, 95:9631-9636). Paradoxically, the ablation of the NF-L gene enhanced the pathological effects of the mutant SOD-1 transgene on primary sensory neurons.

The severe neuropathic effects that result from low level expression of a mutant NF-L transgene (Lee et al. Neuron, 1994, 13:975-988) contrast with the mild neuropathic effects that result from overexpression of the wild-type NF-L (Xu et al., Cell, 1993, 73:23-33) transgene, thus indicating that a mutation in the NF-L transgene markedly enhances the neuropathic effects of the transgene in transgenic mice. This mutant NF-L transgene, however, contained two separate mutations, namely, a leucine-to-proline point mutation in the rod domain of the protein and a 36 bp c-myc tag that was appended to the carboxyl terminus of the protein. The c-myc tag was added in order to mark the NF-L protein from the transgene and distinguish it from the wild-type NF-L protein encoded by the endogenous NF-L gene of the mouse. The leucine-to-proline point mutation in the rod domain was intended to create a dominant disassembling subunit that leads to the disassembly of all NFs in the cell (Gill et al. J. Cell Biol., 1990, 111:2005-2019). Although the neuropathic effects of the transgene were attributed to the point mutation in the rod domain, this interpretation was not supported by a close examination of degenerating motor neurons. Close examination showed that expression of the mutant NF-L subunit did not lead to a granular disintegration of NFs, as characteristic of the dominant disassembly phenotype (Gill et al. J. Cell Biol., 1990, 111:2005-2019), nor prevent the accumulation of assembled NFs, admixed with mutant protein, in cell bodies and dystrophic neurites of the degenerating motor neurons. The inability of the mutant protein to disrupt NFs in degenerating motor neurons indicates that the mutation does not have a dominant disassembly phenotype in vivo and that the accumulation of NFs are, most likely, the result rather than the cause of the degenerative state of motor neurons. Furthermore, the inability of the point mutation to alter assembled NFs also negates the role of this mutation in mediating neuropathic effects. Hence, there must be an alternative explanation accounting for the enhanced neuropathic effect of the mutant NF-L transgene on motor neurons of transgenic mice.

It is now believed that the enhanced neuropathic effects of the mutant NF-L transgene on motor neurons of transgenic mice (Lee et al. Neuron, 1994, 13:975-988) are not due to the point mutation in the rod domain of the protein, but rather to the second mutation created by insertion of the c-myc tag onto the carboxyl terminus of the protein. While the addition of the c-myc tag does not alter the ability of a NF protein subunit to assemble into filaments (Gill et al. J. Cell Biol., 1990, 111:2005-2019), the placement of the 36 bp c-myc tag at the junction between the coding region and 3′UTR of the NF-L cDNA generates a mutant NF-L mRNA that may have altered biological properties. The discovery that the c-myc mutation in the NF-L transcript was inadvertently inserted into the major stability determinant of the transcript supports this view (Ca\check{n}ete-Soler et al., J. Biol. Chem., 1998, 12650-12654 and Ca\check{n}ete-Soler et al., J. Biol. Chem., 1998, 12655-12661). Thus, the neuropathic effects are believed to be due to the c-myc mutation in the NF-L mRNA. Further, expression of the mutant NF-L mRNA, not the mutant NF-L protein, is believed to mediate the neuropathic effects of the mutant transgene in transgenic mice. This discovery has profound implications regarding the pathogenesis of motor neuron degeneration in transgenic mice and on potential treatments and cures of motor neuron diseases.

The biological effects of a c-myc mutation in the NF-L transcript and the ability of NF-L mRNA, not NF-L protein, to mediate neuropathic effects have now been determined. In these experiments, expression of the NF-L transgene with only the c-myc mutation was found to have profound disruptive effects on motor neurons of transgenic mice. Moreover, similar neuropathic changes on motor neurons were reproduced in mice bearing a transgene in which the 3′UTR and c-myc mutation of NF-L was appended to a GFP reporter protein. The latter study shows that the neuropathic effects of the mutant NF-L transgene are due to elements in the NF-L transcript, not to the expression of NF-L protein. Further, a less severe form of motor neuron degeneration was seen in mice bearing a chimeric transgene in which the NF-L 3 UTR was appended to the GFP reporter gene. These findings confirm the presence of elements in the NF-L 3′UTR with neuropathic effects on motor neurons of transgenic mice and indicate the neuropathic effects are enhanced by insertion of the c-myc mutation in the transgene. The results indicate that the milder form of motor neuron degeneration in mice bearing a wild-type NF-L transgene (Xu et al., Cell, 1993, 73:23-33) can also be attributed to neuropathic effects of an element in the NF-L 3′UTR. Finally, biochemical studies now show that similar stability determinants are present in the 3′UTR of the NF-L and NF-H transcripts, thus indicating that motor neuron degeneration from overexpression of a NF-H transgene (Cote et al., Cell, 1993, 35-45) can be attributed to the neuropathic effect of a common element in the NF-H and NF-L 3′UTRs. In summary, it has now been demonstrated that motor neuron degeneration in all three transgenic models arising from expression of different NF transgenes (Xu et al., Cell, 1993, 73:23-33; Cote et al., Cell, 1993, 35-45; Lee et al., Neuron, 1994, 13:975-988) arises from expression of NF transcripts. Further, the neuropathic effects of NF transcripts are due to cis-acting elements that regulate the stabilities of the transcripts and bind common trans-activating factors.

The identification of common cis-acting elements in the 3′UTR of NF mRNAs that lead to motor neuron degeneration in transgenic mice provides important evidence relating to pathways and components thereof which mediate the neuropathic effects. A prime candidate component is the ribonucleoprotein (RNP) complex in brain extracts which binds to the cis-acting element in the 3′UTR of NF-L and NF-H mRNAs that stabilize the transcripts and confer neuropathic effects in transgenic mice. The RNP complexes contain a novel 43 kDa neurofilament mRNA binding protein, referred to as NFRBP-1, that also binds directly to the cis-acting element in the 3′UTR of NF-L and NF-H mRNAs. Insertion of the c-myc mutation into the stability determinant of the NF-L transcript alters the binding of RNP complexes and NFRBP-1 to the stability determinant and disrupts the ability of the stability determinant to regulate the stability of the transcript. Thus, the binding of NFRBP-1 to the NF transcript is closely associated with the stabilization of the transcript and with the enhanced neuropathic effects on motor neurons of transgenic mice that result from presence of the c-myc mutation in the NF-L transgene. The identification of NFRBP-1 and its interactions with other components of neuronal cells and tissue are believed to represent a key link in the pathways leading to motor neuron degeneration in transgenic mice and to motor neuron disease.

### SUMMARY OF THE INVENTION

An object of the present invention is a neuropathic RNA element that produces motor neuron degeneration and that comprises a portion of wild-type neurofilament L mRNA.

Another object of the present invention is a method of identifying components of specific RNA-protein complexes that bind to a neuropathic element and prevent neuropathic effects when expressed with the neuropathic element in neuronal cells and tissues and thus prevent motor neuron degeneration which comprises expressing the neuropathic element of the present invention in a first population of neuronal cells or tissues; contacting said first population of neuronal cells or tissues with a candidate component of a RNA-protein complex; determining a first level of neuropathic effects in said first population of cells or tissues where the candidate component of a RNA-protein complex has been expressed; determining a second level of neuropathic effects in a second population of neuronal cells or tissues that lack expression of the candidate component of the RNA-protein complex; and comparing the level of neuropathic effects in said first and second population of cells or tissues, wherein a decrease in the level of neuropathic effects is indicative of the ability of the candidate component of the RNA-protein complex to prevent motor neuron degeneration.

### DETAILED DESCRIPTION OF THE INVENTION

Motor neuron diseases are a group of disorders characterized clinically by weakness and variable wasting of affected muscles without change in sensory function. A number of theses disorders, including Werdnig-Hoffman disease and Kugelberg-Welander syndrome occur in infants or children. The majority of motor neuron diseases, however, occur sporadically in adults between 30 and 60 years of age. There is a degeneration of the anterior horn cell in the spinal cord, the motor nuclei of the lower cranial nerves, and the corticospinal and corticobulbar pathways. These disorders are progressive and usually fatal within 3 to 5 years; death usually results from pulmonary infection. Riluzole, which reduces the presynaptic release of glutamate has been suggested to slow the progression of amyotrophic lateral sclerosis. Otherwise, there is currently no specific treatment for these disorders.

Molecular biology has shown that highly homologous sets of genes regulate the development, differentiation and function of the nervous system in widely divergent species, from fruit flies and round worms to higher vertebrates, including man. Therefore, the identification of genes or gene products causing motor neuron degeneration in mice has immediate diagnostic and therapeutic applications in human disease. It is believed that very similar sets of genes regulate the homeostasis of motor neurons and are causally related to motor neuron degeneration in human disease, such as amyotrophic lateral sclerosis and spinal muscle atrophy. Similar patterns of gene dysfunction may underlie the degeneration of different subsets of neurons in other neurodegenerative diseases.

The direct and indirect effects of altered neurofilament (NF) expression on neuronal degeneration in transgenic mice indicate that pathways in NF expression interact with pathways that maintain the homeostasis of different subsets of neurons. Recently, it has been shown that these pathways are not necessarily related to the expression of NF proteins as previously thought, but rather to the expression of NF mRNAs and to the ribonucleoprotein (RNP) complexes that mediate the processing of neuronal RNA (Ca\check{n}ete-Soler et al., J. Biol. Chem. 1998, 273:12650-12654; and Ca\check{n}ete-Soler et al., J. Biol. Chem., 1998, 273:12655-12661).

A cis-acting element at the proximal edge of the 3′UTR of the mouse NF-L mRNA and cognate trans-acting factors from brain extracts have now been identified and implicated in the pathogenesis of motor neuron degeneration in transgenic mice. Based upon experiments described herein, it is now believed that the trans-acting factors not only regulate the expression of NF transcripts but also the expression of gene products maintaining the homeostasis of motor neurons. Expression of cis-acting elements in a NF-L transgene alters the trans-acting factors in motor neurons and disrupt the expression of gene products that maintain motor neuron homeostasis, thereby leading to motor neuron degeneration.

A mouse NF-L transgene was constructed with a 36-bp c-myc tag inserted into the stability determinant of the NF-L mRNA. The transgene was placed behind a strong constitutive promoter, hCMV, and microinjected into the mouse germ line. Nine founder mice (out of 67 pups) were recovered. Two founder mice (pups A and B) were born in an agonal state with markedly distended abdomens. Examination of the intestines revealed extensive dilatation of the midgut in pup A and, to a lesser extent, in pup B. No specific sites of intestinal perforation were identified. Milk products were not present, but fecal content were observed throughout the intestines, indicating that the intestinal dilatation was not due to complete obstruction of the alimentary canal.

Microscopic examination showed that intestinal dilatation was associated with a marked depletion of neurons from the enteric nervous system when the population of enteric neurons was visualized by their immunoreactivity. Multiple sections of midgut revealed either an absence or paucity of neurons in the dilated and thinned intestinal walls of the transgenic pups, compared with non-transgenic littermate controls. The aganglionic and hypoganglionic (loss of >50%) segments of midgut differed only in the extent of neuronal loss. Residual neurons were only observed in the myenteric plexus, although neurons of the myenteric and submucosal plexi were seen in controls. Residual enteric neurons did not display any distinctive pathological features and were difficult to identify with certainty on stained sections.

The effect of this transgene on the development of other neurons, specifically motor neurons, in these animals was also examined. Microscopic examinations at multiple levels of spinal cord revealed a vacuolar degeneration of anterior horn cells in the transgenic pups. The perikarya of altered motor neurons were filled with vacuoles of variable sizes, irregular shapes and sharp borders. Vacuolar degeneration was seen in most anterior horn cells at all levels of spinal cord, more so in pup A than in pup B. Vacuolar changes were not seen in other neurons of the spinal cord, nor in any neurons in spinal cords of non-transgenic littermates. A loosening of neuropil in the vicinity of the vacuolated perikarya may have obscured the presence of vacuolar changes in the neurites of motor neurons. A loosening of neuropil was seen in other regions of spinal cord and in spinal cords of non-transgenic newborn mice. The remarkable preservation of nuclear detail, however, attested to the structural preservation of neuronal cell bodies in immersion-fixed, paraffin-embedded tissues. The large, round nuclei of vacuolated motor neurons had sharply defined nuclear borders, displayed a finely granular chromatin pattern and often contained a very prominent nucleoli. The same nuclear details were observed in vacuolated motor neurons of older transgenic mice without loosening of surrounding neuropil.

Immunoreactivities to mouse NF-L and to the human c-myc tag in vacuolated and control motor neurons of newborn mice were tested with increasing concentrations of primary antibodies to mouse NF-L and the c-myc tag. End products were only observed at titers which produced extensive non-specific staining of the tissues. When the spinal cords of newborn mice were examined with highly sensitive antibodies to phosphorylated epitopes on the NF-H and NF-M subunits, immunoreactivity was detected in white matter tracts along the dorsal and ventral surfaces of spinal cord. Focal NF accumulations in cell bodies or nearby neurites of vacuolated motor neurons were not seen in newborn mice or in the spinal cord of older mice. The limited amounts of motor neuron tissues in newborn mice precluded a biochemical assessment of NF-L protein levels by Western blot.

The extensive vacuolar degeneration of motor neurons in newborn mice was associated with perturbations in target organ development. Differences in skeletal muscle development were readily apparent in comparative examinations of muscles at the level of the distal tibia from transgenic and non-transgenic newborn pups. Whereas the muscle of newborn controls was composed of uniform bundles of muscle fibers with occasional central nuclei, the muscle from the transgenic pups A and B contained numerous small cells without myofibrils interspersed among large fibers with myofibrils. The large fibers had large and hyperchromatic central nuclei that were often associated with perinuclear vacuoles. The features resemble those described during muscle development lacking neurogenic input and have been attributed to a persistence and degeneration of primary myotubes and deficiency of secondary myotube development. Differences between transgenic and non-transgenic muscle were less apparent in musculature from the proximal limbs and along the axial skeleton, suggesting that the changes may reflect a preferential involvement of distal musculature or, possibly, a delay in muscle development.

Further, founder mice and transgenic Fl pups were smaller and less active than their non-transgenic littermates. These traits became apparent during the initial 2-3 week period of postnatal development but did not progress and became less apparent after weaning. An abnormal reflex of flexing the limbs when held by the tail was also observed in these mice. The mice did not develop further weakness or paralysis and were sacrificed along with a non-transgenic littermate at 28 days. Microscopic examination revealed a loss of enteric neurons and vacuolar degeneration of motor neurons in the transgenic pups. Stunted growth was also a useful phenotypic marker of some but not all transgenic pups. When subsequently examined for transgene expression, pups with stunted growth had the highest levels of transgene expression in their respective litters.

Abnormal phenotype in the transgenic animals was found to correlate with expression of the mutant transgene. The highest levels of transgene expression were found in newborn pups with dilated and malformed intestine. RNA protection assays showed the highest level of transgene expression in the brain of pup A, slightly less in the brain of pup B, corresponding with the more severe alterations in pup A. Transgene expression was greater than that of endogenous NF-L expression, although the latter is present at very low levels in neonatal rodent brain. Similar levels of NF-L mRNA were noted in newborn transgenic and non-transgenic littermates, indicating that transgene expression did not appear to alter the expression of endogenous NF-L mRNA. Transgene expression was derived from low transgene copy numbers of 2 and 1 in pups A and B, respectively, as estimated by PCR and Southern blot analyses of genomic DNA.

Expression of the transgene in other founder mice and in transgenic lines was variable. A female founder (mouse C) transmitted the transgene to 4 of 7 F1 pups, but the transgene was not expressed and the abnormal phenotype was not detected in this transgenic line. A male founder (mouse D) was unable to transmit the transgene to three litters of F1 pups. When sacrificed at 3 months, focal vacuolar degeneration was observed in anterior horn cells and a low level of transgene expression was detected in brain. Levels of transgene expression also correlated with the severity of neuropathic changes of enteric and motor neurons in the F1 and F2 progeny. Neuropathic changes of enteric and motor neurons occurred in three transgenic lines (from founders E, F and G), including eight transgenic pups from the F1 or F2 generations. Two transgenic pups from founder F were less active and without visible milk products in their stomachs, as customarily seen through the thin abdominal wall of newborn suckling mice. When sacrificed on day 2, the absence of milk products was confirmed by direct examination. Instead, the stomachs and small intestines, but not the abdominal cavities, were distended with air, as if the pups had attempted to suckle but had ingested air instead of milk. Microscopically, there was extensive depletion of enteric neurons in the small intestine and vacuolar degeneration of motor neurons, similar to that described in founder mice A and B.

In several instances, either 1 or 2 newborn Fl pups died during the initial 24 hour postnatal period and were probably cannibalized by the mother so that they were not recovered for genomic typing or examination of the tissues. Nonviability of transgenic pups was also suggested in cross-breeding experiments. Initial cross-breeding of founder mice produced Fl litters with only 1 or 2 viable pups and Fl litters with a higher percentage of non-transgenic pups than anticipated. Subsequent cross-breedings of the same founder mice yielded larger Fl litters with close to the anticipated 75% rate of transgene transmission. Transgenic Fl pups, sacrificed at 14 and 28 days, revealed focal losses of enteric neurons and vacuolar changes of motor neurons. The extent of neuropathic changes and corresponding levels of transgene expression were notably less than those observed in founders A and B in the newborn transgenic F1 pups of founder F. Examinations of other tissue, including the kidneys, from the founder mice and transgenic lines were unremarkable.

The effects of a c-myc insert on NF-L mRNA stability in a neuronal cell line were also examined. A full-length NF-L cDNA (NF-L/wt), a cDNA in which 23 bp of distal coding region and 45 bp of proximal 3′UTR were deleted (NF-L/del) and a cDNA containing a 36 bp c-myc insert between the coding region and 3′UTR (NF-L/c-myc) were constructed. The NF-L cDNAs were then placed behind a Tn-10 tetracycline-inducible promoter and stably transfected into a neuronal cell line containing the tTA transactivator cDNA under control of an autoinducible promoter. The system was shown to be highly inducible when tested with a luciferase reporter gene, generating 1,000-fold increases (and decreases) of luciferase activity in the 48 hour interval after withdrawal (and readdition) of tetracycline.

Stability of mRNAs from the NF-L/wt, NF-L/del and NF-L/c-myc cDNAs were compared by inducing transgene expression for 72 hours in the absence of tetracycline, then measuring mRNA levels at varying timepoints after the readdition of the ligand. The NF-L transcript is stabilized by either deleting the entire binding site of the stability determinant (NF-L/del) or by inserting a c-myc tag between the 3′CR and 3′UTR components of the binding site (NF-L/c-myc). The insertion of the c-myc tag is almost as effective as the full deletion in disrupting the function of the determinant.

Experiments were then performed to determine whether the effect of the 36-bp c-myc insert was due to the c-myc sequence per se or to the context of its placement in the stability determinant. The stability of NF-L mRNAs were assessed when the c-myc insert was placed in exon 1 (NF-L/c-myc/BglII) or in the distal 3′UTR (NF-L/c-myc/EcoRI). In both instances, the presence of the c-myc insert did not alter the stability of the transcript. The stability of the NF-L transcript was enhanced, however, when the c-myc tag was inserted into the stability determinant (NF-L/c-myc).

Thus, insertion of the c-myc tag into the stability determinant of the NF-L mRNA appears to alter the stability of the transcript, not due to the presence of the c-myc sequence, but rather due to the placement of a mutation within the stability determinant. Further support of this is derived from the demonstration that the 36 bp c-myc tag in exon 1 does not gel-shift an RNP complex. Instead, placement of the c-myc tag at the end of the coding region alters the RNP complexes that form on the major stability determinant of the transcript.

Gel-shift and cross-linking assays were also undertaken to compare the complexes that form when brain extracts containing RNPs are incubated with probes of wild-type and mutant gene sequences. The RNP complexes that assemble on an RNA probe of NF-L composed of the 23 nt of 3′CR and 45 nt of proximal 3′UTR consist primarily of a set of bands that is competed away by poly(C) homoribopolymers, enhanced in the presence of poly(U) and has been referred to as the C-binding complex (Ca\check{n}ete-Soler et al., J. Biol. Chem., 1998, 273:1265512661). A similar C-binding complex with a slightly different pattern of electrophoretic migration forms on the probe with a c-myc tag insert between the 3′CR and 3′UTR. While the C-binding complex on the mutant probe is also competed with poly(C) and enhanced in the presence of poly(U), a large percentage of the complex has a slower rate of electrophoretic migration, as if forming a larger aggregate. The formation of an additional slower-migrating component in the C-binding complex on the mutant probe was consistently observed in six gel-shift assays using three different preparations of brain extract that were either freshly prepared or retrieved from storage at −80° C. Formation of slower- and faster-migrating components of the C-binding complex on the mutant probe was observed when gel-shift assays were conducted with 160, 80, 40, 20 or 10 μg of protein. In all instances, at least 35% of radioactivity of the C-binding complex was present in the slower-migrating band.

Insertion of a c-myc tag between the coding region and 3′UTR of the NF-L probe also led to an enrichment of a slow migrating set of bands on the mutant probe that is competed with poly(U) and has been referred to as the U/A-binding complex (Ca\check{n}ete-Soler et al., J. Biol. Chem., 1998, 273:12655-12661). The U/A-binding complex tends to aggregate into a slow-migrating band when assembled in the presence of poly(C). Small amounts of the U/A-binding complex also form on the wild-type probe, but always at lower levels than that formed on the mutant probe.

When the complexes that form on radioactive probes from brain extracts are cross-linked by UV irradiation, digested and examined on denaturing SDS gels, radioactivity from the wild-type and mutant probes are present in a major 43-kDa polypeptide and a minor 80-kDa polypeptide. Since the cross-linking to the 43-kDa polypeptide is competed with poly(C) and the cross-linking to the 80-kDa polypeptide is competed with poly(U), they have been interpreted as core-binding components of the C-binding and U/A-binding complexes, respectively (Caj\check{n}ete-Soler et al., J. Biol. Chem., 1998, 273:12655-12661). Although radioactivities from the wild-type and mutant probes are cross-linked to the same core-binding polypeptides, there are small differences in the amounts of cross-linked radioactivity, especially when formation of the C- or U/A-binding complexes are competed with poly(C) or poly(U). For example, cross-linking to the 80-kDa polypeptide is enhanced in the presence of poly(C) when the wild-type probe is used, but not when the mutant probe is used. Likewise, the addition of poly(U) or poly(A) enhances the cross-linking to the 43-kDa polypeptide from the wild-type probe, but not from the mutant probe. The enhanced cross-linking to core binding polypeptides when formation of C- and U/A-binding complexes are competed, is a characteristic feature of wild-type probes as described by Ca\check{n}ete-Soler et al., J. Biol. Chem., 1998, 273:12655-12661. The lack of a corresponding enhancement in the cross-linking to core binding components from the mutant probe was consistently observed.

Cross-linkage of radioactivity to the 43-kDa polypeptide from the mutant probe was also less than from the wild-type probe. To determine whether the reduction in cross-linkage was due to differences in probe concentration, cross-linkage studies were conducted with increasing amounts of probes that were diluted to the same specific radioactivities. The results indicate that the c-myc mutation leads to a 2-fold reduction in cross-linkage to the 43-kDa polypeptide over a wide range of probe concentrations.

Additional experiments were conducted to confirm the view that neuropathic effects on motor neurons of transgenic mice did not result from expression of protein by the transgene, but rather from expression of mRNA by the transgene, more specifically, from expression of an element in the 3′UTR of the NF-L transgene. Studies were also conducted to show that the presence of the c-myc mutation in the 3′UTR of the NF-L transgene enhanced the neuropathic effects of the transgene on motor neuron degeneration. These results were accomplished by constructing chimeric transgenes in which the 3′UTR of green fluorescent protein (GFP) was replaced with wild-type 3′UTR of mouse NF-L (GFP/NF-L 3′UTR) or with a mutant 3′UTR of mouse NF-L (GFP/c-myc/NF-L 3′UTR) in which a 36-bp c-myc tag was inserted at the proximal edge of the 3′UTR. (Ca\check{n}ete-Soler et al., J. Neurosci., 1999, 19:1273-1283). In both instances, junctional sites were sequenced to confirm the presence and integrity of stop codons at the end of the GFP coding sequences.

The NF-L sequence was placed in the 3′UTR of an EGFP reporter transgene, with experimental (EGFP/NF-L and EGFP/NFL/c-myc) and control (EGFP/wild-type) transgenes containing identical cDNA encoding the EGFP protein driven by an hCMV promoter (Stratagene, Inc., La Jolla, Calif.). The 3′UTR of commercially available EGFP cDNA was either unaltered (EGFP/wild-type) or replaced by NF-L sequence (EGFP/NF-L) or by NF-L sequence with a 36 nucleotide c-myc insert (EGFP/NF-L/cmyc). NF-L sequence encompassed the entire 3′UTR and adjacent coding region and included binding sites of multiple transacting factors. Stop codons were inserted at junctional sites of the chimeric constructs to assure that NF elements remained untranslated. Transfections were undertaken to verify that NF elements in the chimeric transgenes were transcribed but not translated. Transcripts of transgenes with NF sequence in the 3′UTR (EGFP/NF-L and EGFP/NF-L/c-myc) generated RT-PCR fragments of expected sizes using EGFP and NF-L primers. Experimental and control transgenes expressed similar-sized EGFP proteins. Moreover, translation of the c-myc tag was not detected in the 3′UTR of the chimeric transgene (EGFP/NF-L/c-myc), but was readily immunoblotted when present in the coding region of the parent NF-L transgene.

Microinjections of transgenes into mouse zygotes generate 5 EGFP/wild-type, 8 EGFP/NF-L and 4 EGFP/NF-L/c-myc founder mice out of total 48, 108, and 154 pups, respectively. An additional 4 EGFP/NF-L/c-myc pups dies shortly after birth (0 to 2 days). The dead pups did not reveal any obvious phenotype. Their tissues were incompletely recovered and were insufficiently preserved for neuropathological examination. A similar high incidence of perinatal deaths was also observed among transgenic pups bearing a NF-L transgene with the same c-myc mutation (Ca\check{n}ete-Solar et al. 1999).

The 8 EGFP/NF-L and 5 EGFP/wild-type founder mice were bred to generate transgenic lines for assessing the neuropathic effects of the chimeric transgene. Since the transgenes were driven by a constitutive hCMV promoter, expression of EGFP protein in blood samples of transgenic pups was used to assess levels of transgene expression in live pups. As a result, relative levels of transgene expression was able to be compared, and transgenic pups with highest levels of the respective transgenes were then inter-bred to optimize the prospects for high-level expression in four lines bearing the chimeric EGFP/NF-L transgene and three lines bearing the EGFP/wild-type control. Founder mice bearing the EGFP/NF-L/c-myc construct expressed only limited amounts of the transgene and expression of the transgene was then further reduced upon transmission of the transgene in transgenic lines.

While similar levels of EGFP protein were detected in blood samples of founder pups bearing either the EGFP/wild-type or EGFP/NF-L transgenes, expression of the transgene led to a distinctive phenotype. Whereas founder pups expressing the EGFP/wild-type transgene were indistinguishable among their litter, founder pups expressing the EGFP/NF-L transgene were smaller and less active than their non-transgenic litter mates. The affected pups had slower and less agile movements. Severely affected pups had partial ptosis of their eyelids. These traits became apparent between 2 and 3 weeks of age and were most prominent in founder pups with the highest levels of transgene expression. The EGFP/NF-L transgenic pups were monitored carefully with the intent of sacrificing the mice prior to death due to natural causes. However, the phenotypic traits did not progress, instead they receded and gradually disappeared during the 4-8 week period of development.

Founder mice exhibiting the EGFP/NF-L phenotype were mated to obtain high level expression in transgenic lines. All F1 and F2 transgenic pups were weighed, examined and monitored for EGFP expression in blood samples at 2, 3, 4, 6 and 8 weeks of age. Parallel studies were conducted on F1 and F2 pups from the control line obtained by mating founder mice with high level expression EGFP/wild-type transgene. Similar levels of EGFP were observed in blood samples from EGFP/NF-L and EGFP/wild-type transgenic lines.

The phenotype observed in founder pups also occurred in F1 and F2 pups of the EGFP/NF-L transgenic line. Comparative weight analyses revealed a marked reduction at 2 weeks of age among the highest EGFP/NF-L-expressing pups. Average weight was reduced by 50% and weights of individual pups were less than half that of non-transgenic pups from the same stock. Average weight of all 2 week old transgenic pups was reduced to a much less extent, indicating that the retarded growth correlated with levels of NF-L/EGFP expression. Likewise, the recovery from growth retardation was also dependent on levels of transgene expression. Although the average weight of pups expressing EGFP/NF-L recovered fully between 2 and 4 weeks, the more severely affected pups had a more prolonged recovery period. Parallel studies on F1 and F2 transgenic pups expressing high levels of EGFP/wild-type transgene showed that their weights did not differ from the weights of non-transgenic pups during the same post-natal period of development.

Transgenic F1 pups expressing high levels of EGFP/NF-L transgene were not only distinguished in the litter by their small size, but also by reduced activity and by abnormal movements and postures at rest. The pups were also less responsive to prodding and moved for only short distances when prodded. They were readily captured by securing and lifting their tails. When suspended, they struggled less than age-matched controls and tended to flex rather than extend their limbs.

The distinctive phenotype from expression of the EGFP/N-L transgene gradually receded during the 4 to 8 week period of development. The disappearance of the phenotype was accompanied by a progressive reduction of transgene expression in blood cells. A decrease in hemotogenous expression of the transgene was also observed among successive litters of founder and F1 breeders as well as in successive F1, F2 and F3 generations of germ-line transmission. The decline in transgene expression in transgenic lines provided a spectrum of offspring with diminishing levels of transgene expression. The decline was temporarily countered by inter-breeding founder mice with F1 mice that exhibited highest levels of EGFP/NF-L transgene expression.

Mice bearing the EGFP/NF-L transgene developed an age-dependent, slowly progressive deterioration of motor function. These changes were also dose-dependent, as they were most apparent in founder and F1 mice with the highest levels of transgene expression. Adult-onset motor impairment was initially observed at 6 to 8 months and became pronounced at 12 months. By 12 months, the mice displayed a scoliotic posture during locomotion and at rest. They had an awkward, waddling gait with limited movement of distal hindlimb phalanges. When placed on a rotorod, they were unable to spread their hindlimb phalanges for the maintenance of balance. Instead, their hindlimb phalanges remained in a fixed, flexed and pronated position. When suspended, they flexed, instead of extending their limbs and did not struggle or reach for proximal surfaces.

To quantitate motor impairment, a rotorod balancing test was conducted on EGFP/NF-L and EGFP/wild-type transgenic mice and on non-transgenic age-matched controls. Non-transgenic mice at 12-14 months of age were tested to establish baseline retention time at varying rotorod rotational speeds. Performance of EGFP/wild-type transgenic mice did not differ from the rotorod retention times of the age-matched controls. However, age-matched EGFP/NF-L transgenic mice were markedly impaired in their ability to cope with the rotorod. Tests were therefore conducted at very slow speeds, far below the threshold levels that challenged age-matched controls. Nevertheless, EGFP/NF-L transgenic mice with age-dependent motor impairment were unable to stay on the rotorod and did not improve their performance with practice. Average rotorod retention times of EGFP/NF-L transgenic mice were markedly reduced compared with EGFP/wildtype mice and non-transgenic controls.

Transgenic mice bearing EGFP/NF-L and EGFP/wild-type transgenes were sacrificed by cardiac perfusion at varying intervals between 2 weeks and 18 months. Similar levels of EGFP immunoreactivity were widely detected in neural and non-neural tissues of EGFP/NF-L and EGFP/wild-type mice during post-natal development. By comparison, tissues of adult mice bearing the EGFP/NF-L transgene revealed less EGFP immunoreactivity. In neural tissues, EGFP immunoreactivity was widely expressed, primarily in neurons, especially large neurons. In spinal cord, the highest levels of EGFP immunoreactivity were consistently observed in large motor neurons of both EGFP/NF-L and EGFP/wildtype transgenic mice. Lower levels of immunoreactivity were present in neurons of the intermediate and posterior horns, in glia, and occasionally in ependyma. High levels of EGFP immunoreactivity persisted in motor neurons in EGFP/NF-L mice at all ages examined, whereas immunoreactivity in glial cells was most apparent during post-natal development.

Structural alterations of motor neurons in EGFP/NF-L transgenic mice were detected by quantitative analysis of myelinated nerve fibers of ventral nerve roots. Transgenic mice with adult-onset motor impairment consistently revealed a loss of large myelinated fibers in L4/L5 ventral roots as compared to age-matched non-transgenic and EGFP/wild-type controls. The changes were specific for motor neurons since myelinated fibers in L4/L5 sensory roots were indistinguishable from those of agematched controls. The loss of large myelinated nerve fibers in ventral roots was not accompanied by a decline in total numbers of myelinated nerve fibers. Therefore, the changes reflected an atrophy of large fibers or their loss and replacement by smaller regenerated fibers.

Neurodegenerative changes were less apparent in the perikarya of motor neurons of EGFP/NF-L transgenic mice. Although not educed in number, motor neurons in lumbar spinal cords of EGFP/NF-L transgenic mice with adult-onset motor impairment showed reduced immunoreactivity to a choline acetyltransferase marker enzyme, but otherwise revealed similar immunoreactivities to EGFP and to each of the NF protein subunits when compared with tissues from age-matched EGFP/wildtype controls. Expression of the EGFP/NF-L transgene did not lead to focal accumulation of NF proteins in perikarya or in immediate surrounding neurites, even in mice with severe impairment of motor function. Similar levels of NF proteins were also observed in Western blots of tissues from EGFP/NF-L and EGFP/wild-type transgenic mice, including sciatic nerve, anterior and posterior nerve roots, spinal cord, and brain.

Focal changes were observed in motor neurons of EGFP-NF-L transgenic mice associated with loss of large myelinated fibers in L4/L5 ventral roots. These neurons showed disparate preservation of cytoplasm, appearing as either vacuolated, watery and swollen, or shrunken and condensed. The changes were observed surrounding well-preserved nuclei and in well-perfused tissues, suggesting an underlying alteration instead of a fixation artefact. These changes were most frequently seen in motor neurons of high-expressing EGFP/NF-L transgenic mice, including post-natal mice with retarded growth and kinetic activity. In addition, motor neurons of EGFP/NF-L transgenic mice were often surrounded by satellite glial cells when compared with motor neurons of EGFP/wild-type controls. These changes were not associated with an increase in EGFP immunoreactivity.

Chimeric transgenes bearing the NF-L 3′UTR with the c-myc mutations were poorly expressed in transgenic mice and could only be detected during post-natal development in blood cells of single female founder pup. Nevertheless, several founder mice displayed transient post-natal growth retardation, similar to the EGFP/NF-L phenotype. At 2 months, one mouse developed progressive weakness and paralysis and was sacrificed during the early course of her first pregnancy. Microscopically, there was extensive vacuolar degeneration of motor neurons throughout the spinal cord and in enteric neurons of the small intestine. The vacuolar neuronal degeneration and involvement of motor and enteric neurons was the same as that produced by a mutant NF-L transgene with an identical c-myc mutation. The vacuolated motor neurons immunoreacted with EGFP but not with the untranslated c-myc element. The only male EGFP/NF-L/c-myc founder mouse failed to transmit the transgene whereas the two other female founder mice of this strain were infertile. When sacrificed at 9 months, two of these three mice showed focal EGFP expression and vacuolar degenerative changes of motor neurons.

These data demonstrate for the first time that neuropathic effects result from expression of EGFP reporter transgene containing untranslated NF-L sequence (EGFP/NF-L and EGFP/NFL/c-myc), but not from a reporter transgene lacking NF-L sequence (EGFP/wild-type). Both functional and structural changes of motor neurons are produced and correlate with levels of transgene expression. The effects on phenotype produced are dose-dependent. Further, the phenotype produced in the transgenic mice expressing high levels of an untranslated NF-L mRNA closely resembled the phenotype reported in mice expressing high levels of an NF-L transgene (Xu et al. 1993). The effects observed were shown to unrelated to alterations of endogenous NF gene expression since expression of the EGFP/NF-L transgene did not alter NF expression in Western blots or lead to accumulation of NF immunoreactivity in motor neurons.

Data from these experiments provide the first direct causal linkage between an alteration in RNA-processing and a neurodegenerative state in transgenic mice. At the same time, the identification of a neuropathic element in NF-L 3′UTR and the cognate RNA-binding factors provide key biochemical components that can be used to identify other protein or nucleic acid linkages, also referred to herein as specific RNA-protein complexes, in the pathway leading to motor neuron degeneration.

The present invention is, therefore, a neuropathic RNA element that comprises a wild-type neurofilament L mRNA. When present, this element produces motor neuron degeneration. Based on this finding, the present invention also relates to methods of identifying specific RNA-protein complexes that can bind this neuropathic RNA element and thus prevent development of motor neuron degeneration. In this method, the neuropathic element of the instant invention is expressed in a first population of neuronal cells or tissues; the first population of neuronal cells or tissues is contacted with a candidate component of a RNA-protein complex; a first level of neuropathic effects is determined in said first population of cells or tissues where the candidate component of a RNA-protein complex has been expressed; a second level of neuropathic effects is determined in a second population of neuronal cells or tissues that lack expression of the candidate component of the RNA-protein complex; and the level of neuropathic effects in said first and second population of cells or tissues is compared, wherein a decrease in the level of neuropathic effects is indicative of the ability of the candidate component of the RNA-protein complex to prevent motor neuron degeneration. In the context of the present invention, “neuropathic effects” that are determined in the cells or tissues would include, but not be limited to, loss of cells in the second population of cells compared to loss of cells in the first population of cells; preferential occurrence in the second population of cells relative to the first population of cells of neuropathic changes including but not limited to loss or diminution in size or number of neuritic processes, vacuolar changes or accumulation of inclusions in the cytoplasm or nucleus, condensation of nuclear chromatin or peripheral displacement of the nuclei; lessening of motor dysfunctional state in mice with the first population of tissues compared to mice with the second population of tissues; greater loss of motor neurons or neuropathic changes of motor neurons of mice with second population of tissues compared to mice with the first population of tissues; greater loss or atrophy of motor nerve fibers in ventral roots of mice with second population of tissues compared to mice with first population of tissues; and larger amount of gliosis in spinal cord anterior horns of mice with second population of tissues compared with mice with first population of tissues.

The following nonlimiting examples are provided to further illustrate the present invention.

### EXAMPLES

### Example 1

### Construction of Mutant NF-L cDNAs

A full-length mouse NF-L cDNA as described by Ca\check{n}ete-Soler et al., J. Biol. Chem., 1998, 273:1650-12654), in the HindIII/XbaI polylinker sites of pSK+ (Stratagene, La Jolla, Calif.), was used as a PCR template to insert a 36 bp c-myc insert immediately upstream of the stop codon. Overlapping sense and antisense primers to the c-myc insert (upper case), stop codon (underlined) and NF-L sequence (lower case) were synthesized as follows: CTCATTTCTGAAGAGGACTTGATTtgagccctattcccaactattcc(sense; SEQ ID NO:1) and TTCAGAAATGAGCTTTTGCTCCATatctttcttcttagccacc (antisense; SEQ ID NO:2). PCR fragments of upstream (1.7 kb) and downstream (0.5 kb) NF-L sequence were generated using primers to the pSK+ vector sequence flanking the HindIII and XbaI restriction sites. A full-length NF-L cDNA with 36 bp c-myc insert was then generated by PCR using the same flanking primers and the 1.7 kb upstream and the 0.5 kb downstream NF-L PCR fragments as template. The 2.2 kb PCR fragment was gel-excised, cut with HindIII and XbaI and ligated into the HindIII/XbaI sites of a pRC/CMV expression vector (InVitrogen, San Diego, Calif.). The integrity of the NF-L cDNA, c-myc insert and stop codon was confirmed by sequencing both strands of the final construct.

NF-L cDNAs were constructed with the same c-myc insert inserted into BglII site (+828) of exon I (NF-L/c-myc/BglII) or into the EcoRI (+2055) in the distal 3′UTR (NF-L/c-myc/EcoRI). In each instance, sense and antisense oligonucleotides containing the 36 bp c-myc sequence flanked by BglII or EcoRI restriction sites were synthesized, annealed, cut, ligated into the respective restriction sites of the NF-L cDNA and sequenced to determine the orientation of the c-myc insert. Insertion of the c-myc sequence into the BigII site did not alter the open reading frame of the cDNA. The integrity of all constructs was confirmed by sequencing.

The NF-L/wt and NF-L/c-myc cDNAs were converted into templates for RNA probes by PCR using primers that bracketed the 23 bp of 3′CR and 45 bp of 3′UTR and with the T7 promoter sequence appended to the sense primer. The same strategy was used to construct a control c-myc probe with the 23 bp of upstream and 45 bp of downstream sequence that flanked the c-myc sequence of the BglII site (+828) in exon I, using the NF-L/c-myc/BglII cDNA as template.

### Example 2

### Determination of mRNA Stability

cDNAs with wild-type sequence (NF-L/wt), with the stability determinant deleted (NF-L/del) and with c-myc mutations (NF-L/c-myc, NF-L/c-myc/BlgII, and NF-L/c-myc/EcoRI) were placed into the HindIII/XbaI polylinker sites of a pRC/RSV vector (Invitrogen, San Diego, Calif.) in which the RSV promoter had been replaced with the heptamerized Tn-10 tet operator sequence, as previously described by Ca\check{n}ete-Soler et al., J. Biol. Chem., 1998, 273:12650-12654. The modified vectors (NF-L/wt/tet, NF-L/del/tet and NF-L/c-myc/tet) were transfected into Neuro 2a cells containing a tTA transactivator cDNA with an autoinducible promoter. Cells with transactivator and inducible target transgenes were selected by growth in Zeomycin and Neomycin, respectively, and the presence of the transgenes was monitored by PCR. Multiple clones (>100) with both transgenes were pooled. mRNA was assayed by ribonuclease protection assay and levels of NF-L mRNA were normalized to those of β-actin mRNA in transfected cells. Radioactivity was quantitated by phosphoimager.

Transient transfections were conducted to compare the effects of the c-myc insert when placed in the stability determinant (NF-L/c-myc/tet), in exon 1 (NF-L/c-myc/BglII/tet) or in the 3′UTR (NF-L/c-myc/EcoRI/tet). These vectors were cotransfected with equal amounts of wild-type vector (NF-L/tet) in Neuro 2a cells containing the tTA transactivator expression vector. Expression of the target NF-L cDNAs was activated for 12 hours by growth in the absence of tetracycline and NF-L mRNAs were quantitated at 24 and 48 hours after addition of tetracycline.

NF-L mRNAs in transient transfected cells were quantitated by RT-PCR. RNA was extracted from a Qiagen column and used as template for reverse transcriptase with random hexanucleotides as primers. 20-mer PCR primers were chosen that extended the PCR products across the sites of the c-myc inserts, i.e., from +790 to +925 in exon 1 of NF-L (for NF-L/c-myc/BglII), and from +1701 to +1836 (for NF-L/c-myc) and from +2000 to +2135 (for NF-L/c-myc/EcoR1). Antisense primers were admixed at a 1:50 ratio with ³²P end-labeled primers. Samples were run for 15, 20 and 25 cycles, separated on 5% acrylamide gels and radioactivity in the PCR products from the mutant and wild-type transcripts quantitated by phosphoimager.

### Example 3

### Gel-Shift and Cross-Linking of RNP Components

Full-length RNA probes were uniformly labeled with ³²P-UTP, eluted from acrylamide gels and diluted to 2.5×10⁴ cpm/ul immediately prior to use, as previously described by Ca\check{n}ete-Soler et al., J. Biol. Chem., 1998, 273:12655-12661). Gel-shift and cross-linking assays were conducted with 5×10⁴ cpm of probe and 160 μg protein extracted from rat brain cytosol in 50 mM K acetate, 3 mM Mg acetate, 2 mM dithiothreitol and 20 mM HEPES buffer, pH 7.4, with or without homoribopolymer competitors. RNP complexes on RNA probes were cross-linked by 30 minute exposures on ice at 3 cm under a UV light (4×10⁶J/cm²) and the radioactive polypeptides were denatured by boiling in SDS sample buffer and fractionated by SDS-PAGE. High-speed cytosolic extracts were obtained from rat brain and were used fresh or within a 4-month period of storage at −80° C. Radioactivity in gel-shifted and cross-linked bands were quantitated by phosphoimager.

### Example 4

### Transgenic Mice

The NF-L cDNA with 36 bp c-myc insert and hCMV promoter was excised with XhoI and XbaI and microinjected into fertilized eggs of B6SJF1/J female mice that had been mated with B6SJF1/J males. Genomic DNA was extracted from tails of 14-day pups and used to detect the transgene by PCR and to estimate transgene copy number by Southern blot. PCR primers spanned the sequence between +1708 and +1815 and generated PCR fragments of 108 and 144 bp from the wild-type and mutant sequence. Genomic DNA was cut with SacI (+1350) and HincII (+1814) to generate fragments of 464 and 500 bp from the endogenous NF-L gene and NF-L transgene, respectively. These fragments were separated on a 2% agarose gel and hybridized with radioactive cDNA probes made by random primed labeling of the SacI/HincII fragment.

### Example 5

### Tissue Analyses

Transgenic and non-transgenic littermates were euthanized with CO₂, their brains excised for RNA protection assay and vertebral columns, abdominal contents and hindlimb musculature were dissected to expose the tissue for optimal fixation. The intestine were fixed in situ by immersion in 10% neutral buffered formalin (NBF) for 24 hours at 4° C., washed and stored in phosphate-buffered saline (PBS) and representative sections were dehydrated and embedded in paraffin. Microscopic sections were stained with hematoxylin and eosin (H&E) or immunostained with PGP9.5 to delineate enteric neurons in accordance with procedures described by Karaosmanoglu et al., Anat. Rec., 1996, 244:470-480). Antibodies to PGP9.5 (Biogenesis Inc., Sundown, N.H.) were applied at a 1:1000 dilution for 1 hour at room temperature and visualized using goat anti-rabbit biotinylated antiserum and the avidin/biotin detection system (Vector Labs, Burlingame, Calif.). The chromaphore was then developed with 3,3′-diaminobenzidine tetrahydrochloride (Sigma Chemical Co., St. Louis, Mo.).

After 4 hours of fixation, the vertebral columns of 14-day, 28-day and adult mice were further dissected to expose the spinal cords directly to NBF prior to washing and storing of tissues in PBS. The spinal cords were separated from the vertebral column, dehydrated, and embedded in paraffin. Paraffin-embedded spinal cords were then cut and positioned in paraffin blocks to obtain serial microscopic cross-sections from the cervical to the lumbar cord. Spinal cords were stained with hematoxylin, hematoxylin and eosin (H&E) or immunostained with primary antibodies to NF-L (N5139, Sigma), to the phosphorylated epitopes on NF-H and NF-M (Ta5l described by Lee et al., J. Neurosci., 1987, 7:3473-3488 and Carden et al., J. Neurosci., 1987, 7:3489-3504) and to the human c-myc tag (AB1, Calbiochem). Secondary antibodies were biotinylated anti-rabbit or anti-mouse IgGs.

### Example 6

### Expression of Transgenic and Endogenous NF-L mRNA

Brains from transgenic and non-transgenic littermates were homogenized in 4 M guanidinium thiocyanate and total RNA was extracted and stored at −80° C. in formamide as described by Chomczynski, P. and Sacchi, N., Anal. Biochem., 1987, 162:156-159. Levels of mRNA from the endogenous NF-L gene and from the mutant NF-L transgene were quantitated by RNA protection assay using radioactive antisense RNA probes that spanned the 36-bp c-myc insert (+1770). Templates for the RNA probes were generated by PCR and spanned NF-L sequence from +1525 to +1846, including the 36-bp c-myc insert and T7 promoter sequence that was appended to the antisense primer. A full-length RNA probe was uniformly labeled with ³²P-UTP using T7 polymerase as described by Schwartz et al., J. Biol. Chem., 1992, 267:24596-24600 and J. Biol. Chem., 1995, 270:26364-26369. The probe was then separated by electrophoresis, excised from acrylamide gels, eluted overnight into 0.5 M NH₄ acetate, 0.1% SDS and 1 mM EDTA and precipitated with ethanol as described by Ca\check{n}ete-Soler et al., J. Biol. Chem., 1998, 273:12655-12661. RNA protection assay was undertaken by hybridizing brain RNA (10-20 pg) with the RNA probe (10⁴ cpm), as previously described by Schwartz et al., J. Biol. Chem., 1992, 267:24596-24600). Protected fragments of 212 and 322 bp from the wild-type and mutant mRNA were separated by electrophoresis on 7.5% denaturing acrylamide gels and radioactivity of the protected fragments was detected by autoradiogram and quantitated by phosphoimager.

### Example 7

### Preparation of RNA Probes

Full-length NF cDNAs were used as PCR templates for duplicating sequences in coding region and 3′UTR of each NF subunits and cloning the PCR fragments into pSK+ (Stratagene, La Jolla, Calif.). KpnI and XbaI restriction sites were placed in the flanking sequences of the sense and antisense primers and gel-excised PCR products were cut and cloned into the KpnI/XbaI site of pSK+. Linearized vectors were used as templates for T7 polymerase to generate RNA probes for gel-shift and crosslinking studies. Probes were uniformly labeled with ³²P-UTP, eluted from acrylamide gels and diluted to 2.5×10⁴ cpm/μl immediately prior to use, as previously described by Ca\check{n}ete-Soler et al., J. Biol. Chem., 1998, 273:12655-12661. Probes for competition studies were generated with a 1:1000 dilution of ³²P-UTP for quantitation, a level of radioactivity that was insufficient to gel-shift a detectable band by autoradiography. Point mutations in the binding site of the C-binding RNP complex were introduced into the template of the H-36 probe by a PCR-based methodology in which the mutations were incorporated into sense and antisense primers as described by Fisher, C. L. and Pei, G. K., BioTechniques, 1997, 23:570-574. The integrity of the mutations was confirmed by sequencing the constructs.

### Example 8

### Construction of Chimeric Transgenes

The effects of the C-binding sites in the NF 3′UTRs on transcript stability were tested using a tetracycline-inducible system as described in Example 2 that was modified so that the changes in transcript stability were reflected by the relative activities of a luciferase reporter gene. Previous studies had shown that the luciferase gene was highly responsive to a tetracycline-inducible promoter. Chimeric transgenes were constructed in a pRC vector (Invitrogen, Carlsbad, Calif.) by placing the luciferase reporter gene behind the tetracycline-inducible promoter and replacing the 3′UTR of the luciferase gene with the 3′UTR from the mouse NF-L or human NF-H gene, with and without deletion of the respective C-binding sites. PCR duplications of wild-type and deleted forms of NF 3′UTRs were generated with flanking SacII and XbaI restriction sites. Deleted forms were created by splicing upstream and downstream PCR fragments that lacked the sequence of the C-binding sites, as described by Ca\check{n}ete-Soler et al., J. Biol. Chem., 1998, 273:12650-12654. Chimeric transgenes with wild-type NF 3′UTRs contained the full-length sequences of probe L680 or H544, while the deleted forms lacked the 68 nt sequence of L68 and the 70 nt sequence of H70. The chimeric constructs with wild-type and deleted forms of the 3′UTR are referred to as Luc/NF-L/wt and Luc/NF-L/del (for the NF-L constructs) and Luc/NF-H/wt and Luc/NF-H/del (for the NF-H constructs). The integrity of the 3′UTR, deletions and junctional sites was confirmed by sequencing the chimeric constructs.

### Example 9

### Transfections of Neuro 2a Cells with Chimeric Constructs and Assays of Luciferase Activity

Studies were conducted on Neuro 2a cells containing a tTA transactivator transgene behind a autoinducible promoter (pUHD15.1M) for activation of tetracycline-responsive promoters. Neuro 2a cells were transfected with the luc/NF-L/wt, luc/NF-L/del, luc/NF-H/wt and luc/NF-H/del constructs, selected with G418 and selected clones (>100) were pooled. The presence of pUHD15.1M and chimeric constructs was monitored by PCR. Neuro 2a cells containing the tTA transactivator transgene and the tetracycline-inducible target vectors were grown for 72 hours in the absence of tetracycline to generate high-levels of luciferase activity. At this timepoint (time 0), tetracycline (0.5 mg/ml) was added to inactivate the chimeric transgenes, and the loss of luciferase activity was assessed at 24, 48 and 72 hours. Cell lysates were harvested in triplicate at each timepoint and levels of luciferase activity were measured using a luciferase assay system (Promega, Madison, Wis.) and Lumat luminometer. Parallel studies were also conducted on Neuro 2a cells containing pUHD15.1M and tetracycline-inducible luciferase transgene with unmodified 3′UTR. Means and standard deviations of 4 experiments were determined.

### Example 10

### Expression of Untranslated RNA by a Transgene

The mouse NF-L/wt and NF-L/c-myc cDNAs were used as templates for PCR amplification of sequence between +1482 and +2161 of the NF-L/wt cDNA. BglII and XbaI restriction sites were appended to the sense and antisense primers to facilitate insertion of the BglII/XbaI restriction fragments into the BglII/XbaI sites of the multicloning site of the GFP expression vector, pEGFP-C2 (Clontech). The sense primer also contained an in-frame TAA stop codon immediately downstream of the BglII site. The integrity of the in-frame stop codon was confirmed by sequencing the junctional sites. The transgenes with strong constitutive hCMV promoter and 3′UTRs were excised with AsnI and MluI and microinjected into fertilized eggs of B6SJF1/J female mice that had been mated with B6SJF1/J males.

### Example 11

### Assessing Transgene Expression in Transgenic Mice

Genomic DNA was extracted from tail clippings of 14-day pups and used to detect the transgene by PCR and to estimate transgene copy number by Southern blot. At the same time, pups were weighed and a small amount of blood from the clipped tail was admixed with PBS containing 0.5% formalin. Blood samples were pelleted, smeared on glass slides and the white blood cells were monitored for GFP fluorescence and for expression of immunoreactivity to rabbit anti-GFP antibodies (Clontech) at 1:100, 1:400 and 1:1600 dilutions of primary antisera. The presence and abundance of transgene in genomic DNA were correlated with semi-quantitative estimates of transgene expression in white blood cells, with relative body weights and with the appearance of altered movements or behavior. The above parameters, including transgene expression in white blood cells, were assessed at 3, 4 and 6 weeks in transgene pups.

What is claimed is:

1. An neuropathic RNA element that produces motor neuron degeneration comprising a wild-type neurofilament L mRNA.

2. A method of identifying specific components of RNA-protein complexes that can be used to prevent motor neuron degeneration comprising:
a) expressing the neuropathic element of claim 1 in a first population of neuronal cells or tissues;
b) contacting said first population of neuronal cells or tissues with a candidate component of a RNA-protein complex;
c) determining a first level of neuropathic effects in said first population of cells or tissues where the candidate component of a RNA-protein complex has been expressed;
d) determining a second level of neuropathic effects in a second population of neuronal cells or tissues that lack expression of the candidate component of the RNA-protein complex; and
e) comparing the level of neuropathic effects in said first and second population of cells or tissues, wherein a decrease in the level of neuropathic effects is indicative of the ability of the candidate component of the RNA-protein complex to prevent motor neuron degeneration.


SEQUENCE LISTING (2 sequences)

SEQ ID NO: 1 - DNA, 47 bp, Artificial Sequence
  Description of Artificial Sequence Synthetic
ctcatttctgaagaggacttgatttgagccctattcccaactattcc

SEQ ID NO: 2 - DNA, 43 bp, Artificial Sequence
  Description of Artificial Sequence Synthetic
ttcagaaatgagcttttgctccatatctttcttcttagccacc